(19) United States
(12) Patent Application Publication
(10) Pub. No.: US 2022/0365492 A1
(43) Pub. Date: Nov. 17, 2022

(54) COMPUTER-READABLE RECORDING MEDIUM STORING CONTROL PROGRAM, CONTROL APPARATUS, AND METHOD OF CONTROLLING

(71) Applicant: FUJITSU LIMITED, Kawasaki-shi (JP)

(72) Inventor: Yoshinobu Iimura, Kawasaki (JP)

(73) Assignee: FUJITSU LIMITED, Kawasaki-shi (JP)

(21) Appl. No.: 17/694,734

(22) Filed: Mar. 15, 2022

(30) Foreign Application Priority Data
Apr. 28, 2021 (JP) 2021-076637

(51) Int. Cl. G05B 13/02

(52) U.S. Cl. CPC G05B 13/026; G05B 13/0265

(57) ABSTRACT

A non-transitory computer-readable recording medium storing a control program for causing a computer to execute a process for controlling a system. The process includes, obtaining a result of a target that fluctuates in accordance with control performed by a system, calculating a weight for a control value in accordance with the result, a history of the control value input to the system and a comparison between the result and a predetermined range, calculating the control value based on the result and the weight, and inputting the calculated control value to the system to control the target.

### CROSS-REFERENCE TO RELATED APPLICATION

This application is based upon and claims the benefit of priority of the prior Japanese Patent Application No. 2021-76637, filed on Apr. 28, 2021, the entire contents of which are incorporated herein by reference.

### FIELD

The embodiment discussed herein is related to a computer-readable recording medium storing control program, control apparatus, and method of controlling.

### BACKGROUND

There exists a related-art technique by which control is performed so as to confine control target data within a target range by using a prediction result by a model generated by machine learning or the like.

For example, there has been proposed a model predictive control apparatus that includes a feedback processing unit that feeds back an actual measurement value of a state amount or a control amount of a control target, and corrects, based on the actual measurement value, a search range or a predicted value in an optimum operation amount search unit and an estimated value in an internal state estimation unit. For example, this apparatus sets a calculation range of an operation amount by an operation amount candidate calculation unit based on the actual measurement value of the fed back state amount or control amount, thereby omitting unnecessary arithmetic. This apparatus corrects an operation amount candidate that is an input to a model prediction unit, the control amount that is an output of the model prediction unit, or an optimum control amount that is input to the control target, and further corrects an output from the internal state estimation unit, thereby improving prediction accuracy.

For example, there has also been proposed a control apparatus for an internal combustion engine. This apparatus calculates an optimum operation amount under model predictive control for which a simple plant model is used. This apparatus calculates a history of a predicted value of the control amount in a predetermined period in the past by using a linear plant model from a history of a command value or an actual value of the operation amount in the predetermined period in the past. This apparatus obtains a history of an actual value of the control amount in a past predetermined period to calculate a parameter of a linear parameter-varying (LPV) error function based on a history of the difference between the predicted value and the actual value of the control amount in the past predetermined period. This apparatus corrects the linear plant model by using the LPV error function and calculates the command value of the operation amount for the next step or for a predetermined period after the next step by the model predictive control using the corrected linear plant model.

For example, there has also been proposed a system that includes a blood glucose level sensor, an insulin injection device, and a control unit that predicts a future evolution of the blood glucose level of a patient based on a physiological model and controls the insulin injection device by considering the prediction. The control unit performs a step of automatic calibration of the physiological model by considering a history of the blood glucose level measured by the sensor during a past observation period. At the end of the calibration step, the control unit determines whether the model is satisfactory based on at least one numerical index representing an error between the blood glucose level estimated based on the model and the actual blood glucose level measured by the sensor. When the quality of the model is not satisfactory, the control unit controls the insulin injection device without considering the prediction made based on the model.

Japanese Laid-open Patent Publication No. 2006-172364, Japanese Laid-open Patent Publication No. 2013-142376, and U.S. Patent Application Publication No. 2020/0015738 are disclosed as related art.

### SUMMARY

According to an aspect of the embodiments, a non-transitory computer-readable recording medium storing a control program for causing a computer to execute a process including: obtaining a result of a target that fluctuates in accordance with control performed by a system; calculating a weight for a control value in accordance with the result, and a history of the control value input to the system and a comparison between the result and a predetermined range; calculating the control value based on the result and the weight; and inputting the calculated control value to the system to control the target.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a block diagram schematically illustrating a configuration of a control system;

FIG. 2 is a diagram for explaining an application example of blood glucose level control by insulin administration;

FIG. 3 is a functional block diagram of a control apparatus;

FIG. 4 is a diagram for explaining a case where results of target data do not fall within a target range;

FIG. 5 is a diagram illustrating an example of a weight database (DB);

FIG. 6 is a diagram for explaining a process of a weight learning unit;

FIG. 7 is a diagram for explaining the process of the weight learning unit;

FIG. 8 is a diagram illustrating an example of a plurality of relationships between yᵢₙᵢ and Λ accumulated in the weight DB;

FIG. 9 is a block diagram schematically illustrating a configuration of a computer functioning as the control apparatus;

FIG. 10 is a flowchart illustrating an example of a control process;

FIG. 11 is a flowchart illustrating an example of a weight learning process;

FIG. 12 is a diagram illustrating examples of control results in the case where learning or calculation of a weight for a control value is not performed;

FIG. 13 is a diagram illustrating a comparison between predicted values and the results of the target data in the case where learning or calculation of the weight for the control value is not performed; and

FIG. 14 is a diagram illustrating examples of control results according to the present embodiment.

### DESCRIPTION OF EMBODIMENTS

In the case where the control is performed so as to confine the control target data within the target range by using the prediction result by the model, the control target data is not necessarily confined within the target range. One of the causes of this is that the prediction by the model is incorrect. In this case, in order to confine the control target data within the target range, it is conceivable to refine the model to improve the prediction accuracy. However, for example, in the case of refining the model by a statistical method or the like, a large amount of training data and a large amount of processing are desired in order to improve the prediction accuracy of the model. This causes a problem in that adaptive handling is unable to be performed during the control of the control target.

According to one aspect, an object of the disclosed technique is to adaptively perform control to confine a control target within a specific predetermined range.

Hereinafter, an example of an embodiment according to the disclosed technique will be described with reference to the drawings. Although a case of blood glucose level control by insulin administration will be described as an application example of the disclosed technique in the following embodiment, the application example is not limited to this.

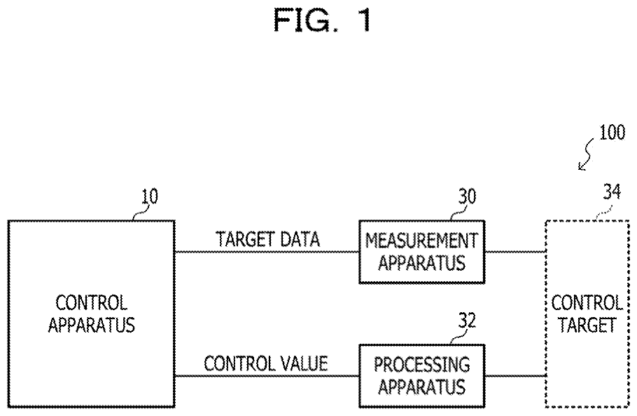

As illustrated in FIG. 1, a control system 100 according to the present embodiment includes a control apparatus 10, a measurement apparatus 30, and a processing apparatus 32. The measurement apparatus 30 measures and outputs control target data 34 (hereinafter referred to as “target data”). Based on a control value calculated by the control apparatus 10, the processing apparatus 32 executes a predetermined process for controlling the control target. By using results of the target data, the control apparatus 10 predicts future target data, and based on results of the prediction, the control apparatus 10 calculates the control value to be input to the processing apparatus 32 so as to confine the target data within a target range. The control target is an example of the “target” of the disclosed technique, and the target range is an example of the “specific predetermined range” of the disclosed technique.

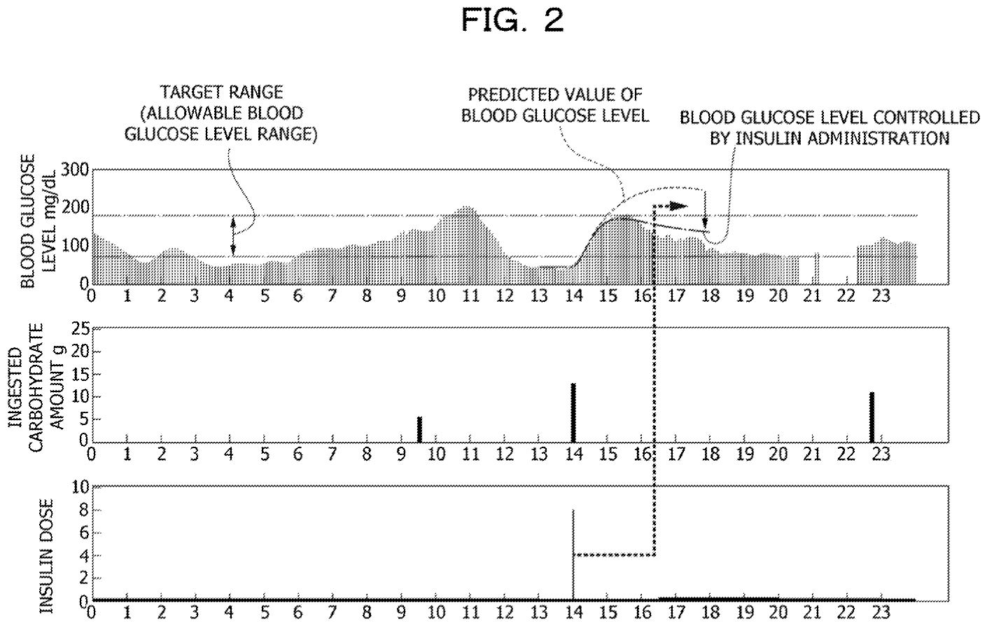

In the case of the application example of the blood glucose level control by insulin administration, the control target is a blood glucose level of a patient, the measurement apparatus 30 is, for example, a blood glucose level measurer that measures the blood glucose level of the patient, and the processing apparatus 32 is, for example, an insulin pump that administers insulin to the patient. The blood glucose level of the patient fluctuates depending on an insulin dose and the like. For example, as illustrated in FIG. 2, in this application example, in the case where the predicted value of the blood glucose level predicted based on the results of the blood glucose level exceeds the upper limit of the target range, control is performed so as to confine the blood glucose level within the target range by decreasing the blood glucose level by administering insulin. In contrast, in the case where the predicted value of the blood glucose level falls short of the lower limit of the target range, control is performed so as to confine the blood glucose level within the target range by increasing the blood glucose level by decreasing the dose of insulin. For example, the control apparatus 10 calculates a control value indicating the insulin dose based on the blood glucose level of the patient measured by the blood glucose level measurer serving as the measurement apparatus 30. Based on the calculated control value, insulin is administered to the patient by using the insulin pump serving as the processing apparatus 32. A bar graph in the upper part of FIG. 2 represents the results of the blood glucose level that is the target data. In this application example, the target range is, for example, a range of an allowable blood glucose level. Although data on the ingested carbohydrate amount that affects the blood glucose level is also included in FIG. 2, description thereof will be omitted herein.

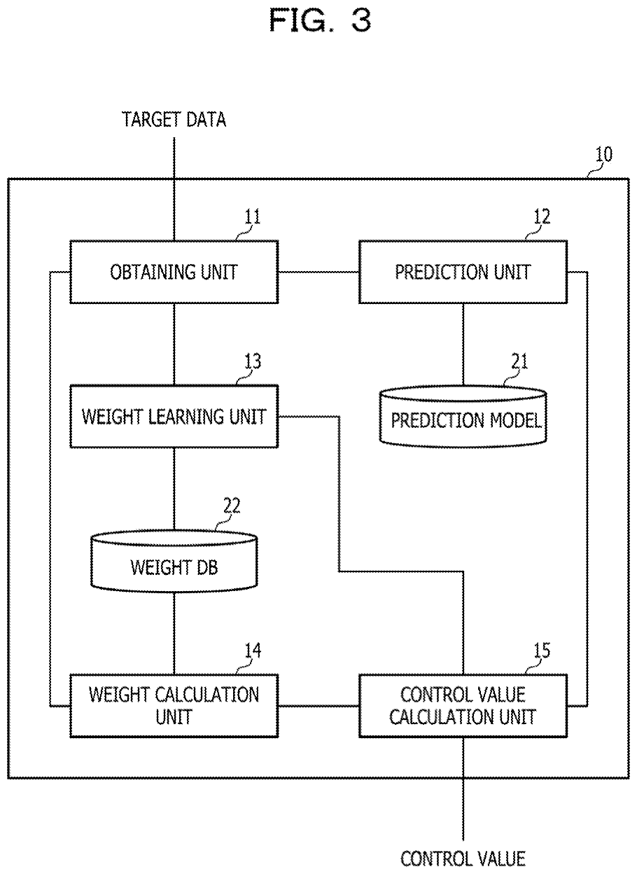

As illustrated in FIG. 3, the control apparatus 10 functionally includes an obtaining unit 11, a prediction unit 12, a weight learning unit 13, a weight calculation unit 14, and a control value calculation unit 15. A prediction model 21 and a weight database (DB) 22 are stored in a predetermined storage area of the control apparatus 10.

The obtaining unit 11 obtains the results of the target that fluctuates in accordance with the control by the control system 100. For example, the obtaining unit 11 obtains the target data measured by the measurement apparatus 30. The obtaining unit 11 transfers the obtained target data to each of the prediction unit 12, the weight learning unit 13, and the weight calculation unit 14.

Based on the prediction model 21, the results of the target data transferred from the obtaining unit 11, and the control value calculated by the control value calculation unit 15, which will be described later, the prediction unit 12 predicts a value of the target data (hereinafter referred to as a “predicted value”) at or after a time at which the control target is controlled. Hereinafter, the time at which the control target is controlled is referred to as a “control time”. According to the present embodiment, the time at which the control value is calculated, the time at which the control value is input to the processing apparatus 32, and the time at which the processing apparatus 32 executes the process of controlling the control target are assumed to be the same, and all these times are referred to as the control time. The prediction model 21 is generated in advance by machine learning such that, in the case where a result and the control value of the target data at a certain time are input, the predicted values of the target data at or after this (current) time are output. For example, the prediction model 21 may output the predicted value under the constraints of the following Expressions (1) and (2).

xₖ₊₁=Axₖ+Buₖ (1)

yₖ=Cxₖ (2)

In the above Expressions, k is an index indicating a time. Hereinafter, the time indicated by the index k is referred to as a “time k”. In the above Expressions, yₖ is a result of the target data at the time k, uₖ is a control value at the time k, and xₖ is a predicted value of the target data at the time k. A, B, and C are parameter matrices determined by machine learning. By using the prediction model 21, the prediction unit 12 predicts a predicted value of the target data at each of a plurality of times at or after the control time. For example, the prediction unit 12 performs a long-term prediction on the target data. For example, the prediction unit 12 sets a cycle of the long-term prediction as h and predicts a predicted value xₖ₊ᵢ (i=0, 1, . . . , h) of the target data at each of times k, k+1, . . . , k+h. The prediction unit 12 transfers the predicted value of the target data that the prediction unit 12 has predicted to the control value calculation unit 15.

The control value calculation unit 15 calculates the control value based on the predicted value transferred from the prediction unit 12 and a weight (details of which will be described later). For example, the control value calculation unit 15 calculates the control value by optimizing an objective function including a term for optimizing so as to confine the predicted value of the target data within the target range and a term for optimizing an input plan of the control value. For example, the objective function may be represented by the following Expression (3).

$${\sum }_{i = 0}^{h} {x}_{k + i}^{T} {Qx}_{k + i} + {\sum }_{i = 0}^{N} {u}_{k + i}^{T} {Ru}_{k + i} \qquad ( 3 )$$

A first term of Expression (3) is a term for optimizing so as to confine the predicted value of the target data within the target range as described above, and a second term is a term for optimizing the input plan of the control value. Last input timing of the input plan of the control value between the times k and k+h is represented by N. For example, in the case where control value is input at the times k, k+j, and k+N, i of the second term is i=0, j, and N. Q is the weight for the predicted value of the target data, R is the weight for the control value, and T represents transposition. The control value calculation unit 15 substitutes the predicted value xₖ₊ᵢ(i=0, 1, . . . , h) of the target data transferred from the prediction unit 12 and the weight R transferred from the weight calculation unit 14 into the objective function of Expression (3) to calculate control values uₖ₊ᵢ that minimize the objective function.

Out of the calculated control values uₖ₊ᵢ, the control value calculation unit 15 outputs a control value uₖ for the time k that is the control time and inputs to the processing apparatus 32 to control the control target. The control value calculation unit 15 transfers the calculated control values uₖ₊ᵢ to the prediction unit 12 so that the calculated control values uₖ₊ᵢ are used for the long-term prediction of the target data at the next control time. The control value calculation unit 15 transfers the control value uₖ input to the processing apparatus 32 to the weight learning unit 13.

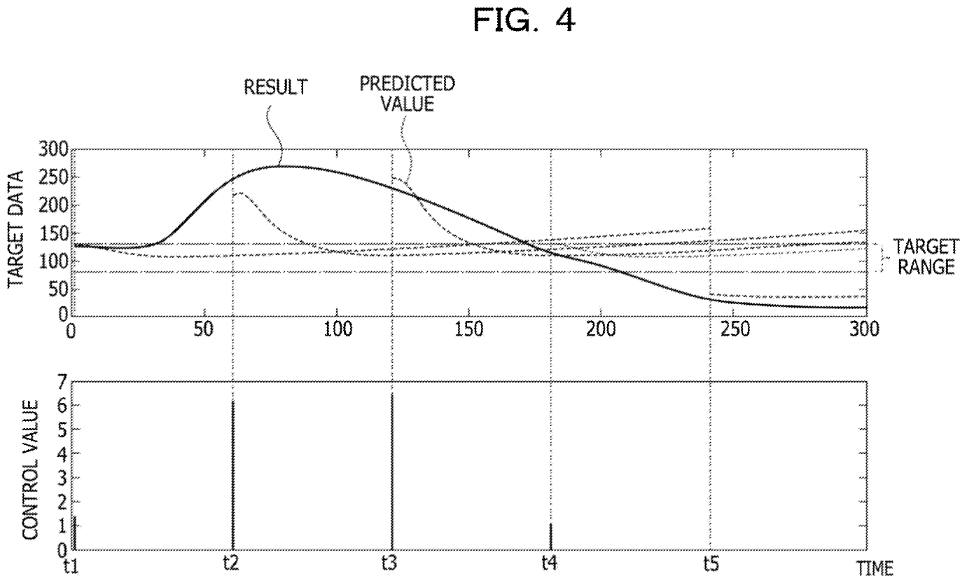

When the control value calculated by the control value calculation unit 15 is not appropriate, as illustrated in FIG. 4, the results of the target data do not fall within the target range. The example of FIG. 4 illustrates an example of the control target for which the value of the target data decreases as the control value increases. For example, this includes an example in which a blood glucose level that is a control target decreases by administration of insulin as in the blood glucose level control by insulin administration. In this example, it is thought that, since the control value input at the time t3 is excessively large, the result of the target data excessively decreases and significantly falls short of the target range. It is thought that the cause of calculation of the control value with which the result of the target data falls outside the target range as described above is that the long-term prediction of the target data is incorrect.

Accordingly, in order to confine the target data within the target range, it is conceivable, as a simply assumed method, to improve the accuracy of the long-term prediction of the target data by the prediction unit 12. For improving the prediction accuracy, for example, it is conceivable to refine the prediction model 21 by using a statistical approach. However, for this approach, a large amount of training data for executing machine learning of the prediction model 21 is desired. For example, in the case of the prediction model 21 constrained by Expressions (1) and (2), the number of parameters to be determined is the total number of the elements of the parameter matrices A, B, and C. For example, when the xₖ is four-dimensional, A has 4×4 elements, B has 4×1 elements, and C has 4×1 elements, and it is desired that a total of 24 parameters be determined. To collect a large amount of training data for determination of such a large number of parameters, a certain period of time is taken. Depending on the control target, in some cases adaptive measures is desired before a sufficient number of pieces of training data are collected. For example, in the application example of the blood glucose level control by insulin administration, in the case where the blood glucose level falls outside the target range on the hyperglycemic side, cerebral infarction, myocardial infarction, gangrene, or the like may be caused by arteriosclerosis. In contrast, in the case where the blood glucose level falls outside the target range on the hypoglycemia side, there is a possibility of the life being directly at risk due to the hypoglycemia. In order to avoid such situations, adaptive measures for confining the target data within the target range are desired.

Accordingly, the control apparatus 10 according to the present embodiment directly adaptively corrects the control value by using an optimization problem of calculating the control value based on the past control results before this control time. For example, the control apparatus 10 corrects the weight for the control value in the optimization, for example, R in the case of the objective function given by Expression (3). As described above, since excessively large or small control value uₖ causes the target data to be out of the target range, uₖ is adjusted through R. For example, in the example of Equation (3), the control apparatus 10 corrects R by using the fact that the uₖ decreases as R increases and the uₖ increases as R decreases. In this case, since the number of parameters to be corrected is one, the adaptive measures may be performed compared to the case where the prediction model 21 is refined by a statistical approach as described above. Hereinafter, each of the weight learning unit 13 and the weight calculation unit 14 related to the calculation of the weight is described in detail.

The weight learning unit 13 learns the relationship between a history of the control value and the result of the target data at a predetermined time and the weight corresponding to the history of the control value and the result. For example, this relationship is, based on the history of the control value and the result of the target data at the predetermined time, for determining the weight for the control value calculated at the time. As the weight defined in this relationship, the weight learning unit 13 uses a weight corrected based on the results of the target data for a predetermined period from a predetermined time. For example, the weight learning unit 13 identifies, at each control time, a weight associated with the history of the control value and the result that are closest to the history of the control value and the result at a time that is a predetermined period before the control time in an already learned relationship. The weight learning unit 13 adds to the already learned relationship a correspondence between a weight obtained by correcting the identified weight in accordance with a comparison between the target range and the results of the target data for the predetermined period from the time before the predetermined period from the control time and the time before the predetermined period from the control time.

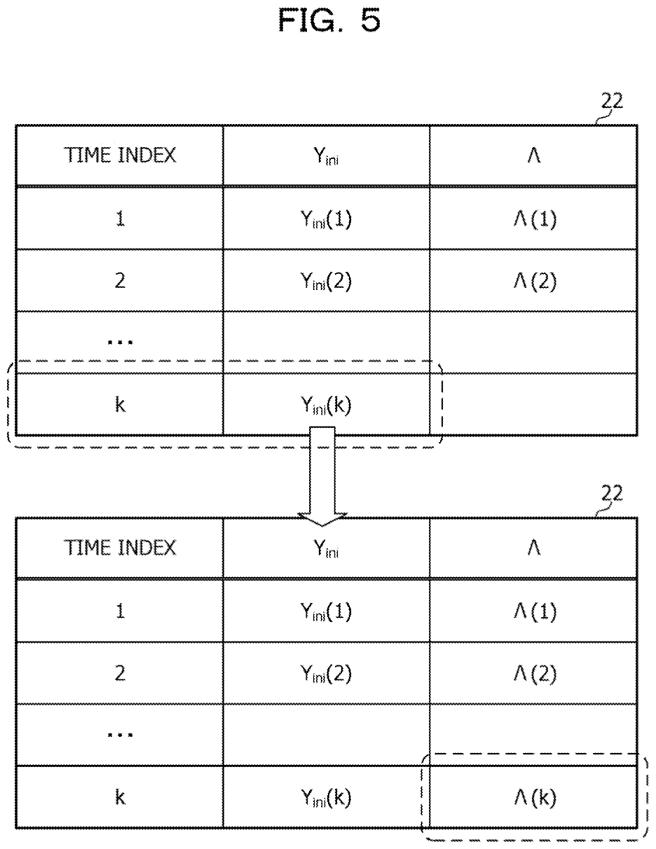

A process of the weight learning unit 13 is described in more detail. The weight learning unit 13 sets, as Yᵢₙᵢ(k), a vector value including values of the result yᵢₙᵢ(k) of the target data at the time k−h being a single cycle (h) before the control time k and the history uᵢₙᵢ(k) of the control value for a predetermined period to the time k−h. A predetermined period of the history of the control value may be, for example, a time length corresponding to three times (k−h−1, k−h−2, k−h−3). In the application example of the blood glucose level control by insulin administration, the predetermined period may be determined in consideration of a duration of insulin effects. For example, in the case where the cycle of the control time is 30 minutes and the duration of insulin effects by a single administration is five hours, a time length corresponding to ten control times may be set as the predetermined period. The weight learning unit 13 sets, as Yₖ, a vector value including values of the result yₖ of the target data at the time k and the history u(k) of the control value for a predetermined period to the time k. A predetermined period of the history of the control value is the same as the above description. The weight learning unit 13 stores Yᵢₙᵢ(k) in the weight DB 22 with Yᵢₙᵢ(k) associated with the index k of the time. FIG. 5 illustrates an example of the weight DB 22. In the example illustrated in FIG. 5, the weight DB 22 stores the weight Λ and Yᵢₙᵢ at the time with Λ and Yᵢₙᵢ associated with the index of the time. A symbol of “Λ (lambda)” is used to distinguish the weight in the weight DB 22 from the weight R used to calculate the control value at the control time. Here, as indicated by a dashed box in the upper diagram of FIG. 5, k and Yᵢₙᵢ(k) are stored in the weight DB 22 in association with each other.

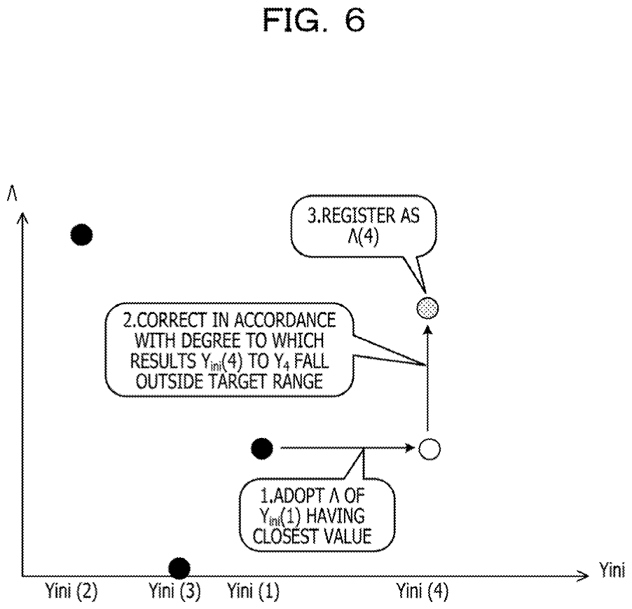

The weight learning unit 13 identifies a weight Λ(ind1) corresponding to Yᵢₙᵢ(ind1) having a value closest to that of Yᵢₙᵢ(k) out of Yᵢₙᵢ stored in the weight DB 22. Here, ind1 is an index of a time corresponding to Yᵢₙᵢ having a value closest to that of Yᵢₙᵢ(k). For example, FIG. 6 illustrates the relationship between Yᵢₙᵢ(k) and Λ(k) when the control time k=4 and Yᵢₙᵢ(k) and Λ(k) for k=1, 2, 3 are already stored in the weight DB 22. In FIG. 6, solid dots represent the relationship between Yᵢₙᵢ(k) and Λ(k) (k=1, 2, 3) already stored in the weight DB 22. In this case, as indicated by an empty dot illustrated in FIG. 6, the weight learning unit 13 identifies Λ(1) corresponding to Yᵢₙᵢ(1) having a value closest to that of the Yᵢₙᵢ(4) and temporarily adopts as Λ(4).

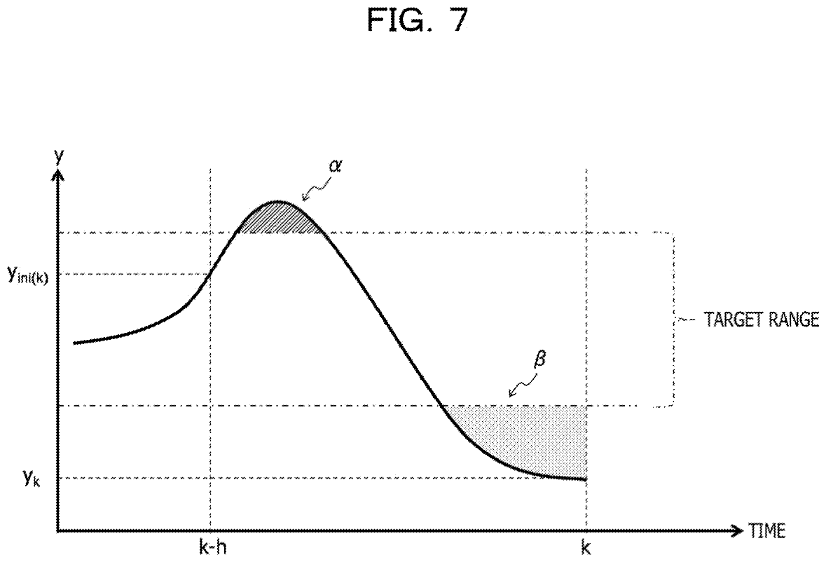

As illustrated in FIG. 7, the weight learning unit 13 calculates, for a period from the time k−h to the time k, an index a indicating a degree to which the results of the target data exceed the upper limit of the target range and an index β indicating a degree to which the results of the target data fall short of the lower limit of the target range. The index α may be a value corresponding to the area of a hatched portion illustrated in FIG. 7. The index β may be a value corresponding to the area of a shaded portion illustrated in FIG. 7. For example, as represented by Expression (4) below, the index α may be a mean square error of the results that exceed an upper limit U of the target range out of the results yᵣ of the target data at each time r in the period from the time k−h to the time k. Likewise, as represented by Expression (5) below, the index β may be a mean square error of the results that fall short of a lower limit L of the target range out of the results yᵣ of the target data.

α=∥yᵣ(yᵣ>U)−U∥ (4)

β=∥L−yᵣ(yᵣ<L)∥ (5)

In Expression (4), yᵣ(yᵣ>U) represents yᵣ that exceeds the upper limit U, and in Expression (5), yᵣ(yᵣ<L) represents yᵣ that falls short of the lower limit L.

The weight learning unit 13 corrects the identified Λ(ind1) based on the calculated indices α and β and calculates Λ(k) to be stored in the weight DB 22. For example, the weight learning unit 13 corrects Λ(ind1) such that the target data decreases in accordance with the magnitude of the index α and corrects Λ(ind1) such that the target data increases in accordance with the magnitude of the index β. As in the application example of the blood glucose level control by insulin administration, when the target data decreases as the control value increases, the weight learning unit 13 calculates Λ(k) by correcting Λ(ind1) so as to decrease the Λ(ind1) to increase the control value as the index α increases. Likewise, the weight learning unit 13 calculates Λ(k) by correcting Λ(ind1) so as to increase Λ(ind1) to decrease the control value as the index β increases.

For example, when α is greater than β (including the case where β is 0), the weight learning unit 13 may calculate Λ(k) after the correction by Expression (6) below. When β is greater than α (including the case where α is 0), the weight learning unit 13 may calculate Λ(k) after the correction by Expression (7) below. When both α and β are 0, for example, when any of the result yᵣ of the target data at each time r in the period from the time k−h to the time k is within the target range, the weight learning unit 13 may set Λ(ind1) to Λ(k) as it is as represented by Expression (8) below.

When α>β≥0,

Λ(k)=Λ(ind)+(α−β)/N1×(0−Λ(ind1)) (6)

When β>α≥0,

Λ(k)=Λ(ind)+(β−α)/N2×(Rmax−Λ(ind1)) (7)

When α=0 and β=0,

Λ(k)=Λ(ind) (8)

Each of N1 and N2 is a normalization constant. Rmax is the maximum value that may be set as the weight R. The calculation method and the classifications of the cases of Λ(k) described above are merely exemplary and may be changed as appropriate in accordance with the characteristics or the like of the control target. For example, in the case of the application example of the blood glucose level control by insulin administration, since there is a significant risk in the case of hypoglycemia, for example, in the case where the target data falls short of the target range, even when α>β, as long as β>0, Expression (9) below may be adopted.

Λ(k)=Λ(ind)+β/N2×(Rmax−Λ(ind1)) (9)

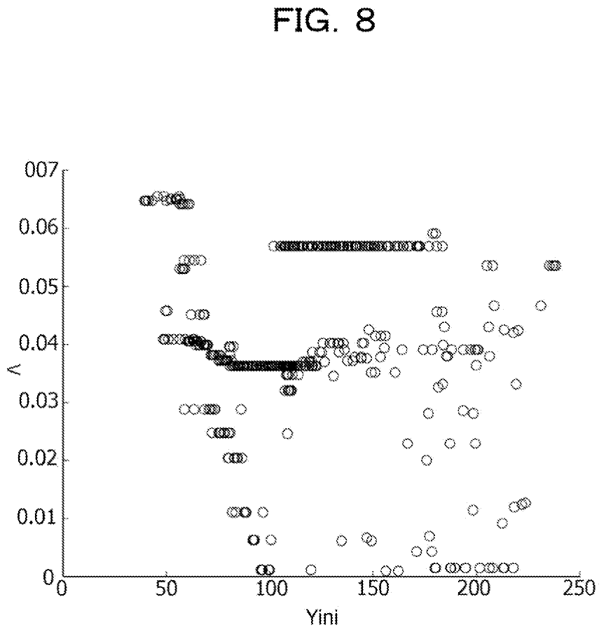

As illustrated in FIG. 6, in the case of the above-described example of the control time k=4, the weight learning unit 13 calculates Λ(4) (shaded dot illustrated in FIG. 6) by correcting the identified Λ(1) (empty dot illustrated in FIG. 6) in accordance with the degree to which the results yᵢₙᵢ(4) to yₖ of the target data fall outside the target range. As indicated by a dashed box in the lower diagram of FIG. 5, the weight learning unit 13 stores the calculated Λ(k) in the weight DB 22 with Λ(k) associated with the index k of the time. When the weight learning unit 13 repeats the above-described process at each control time k, a plurality of the relationships between Yᵢₙᵢ and Λ are accumulated in the weight DB 22. FIG. 8 illustrates an example of the plurality of relationships between Yᵢₙᵢ and Λ accumulated in the weight DB 22. In the example illustrated in FIG. 8, a single dot represents a single relationship between Yᵢₙᵢ and Λ.

It is also conceivable that the relationship between Yᵢₙᵢ and Λ is represented by Λ=f(Yᵢₙᵢ) by using a function f(⋅). However, Yᵢₙᵢ is obtained only with a limited number of trials, and not all continuous values are obtained. It is not clear what kind of function is to be prepared as f(⋅) because there is no presupposed knowledge. When the appropriate function f(⋅) is not used, Λ after the correction is not able to be appropriately calculated. This leads to degradation of control performance. In contrast, as described above, the control apparatus 10 according to the present embodiment corrects Λ for the past Yᵢₙᵢ having the closest value to Yᵢₙᵢ(k) at the control time k based on the results of the target data and stores the relationship between Yᵢₙᵢ and corrected Λ. When the control apparatus 10 repeats this process at each control time k, an arbitrary f(⋅) may be represented and degradation of control performance may be suppressed.

The weight calculation unit 14 calculates the weight corresponding to the history of the control value and the result obtained this (current) time based on the relationship between the history of the control value and the result of the target data in the past and the weight corresponding to the history of the control value and the result in the past stored in the weight DB 22. For example, the weight calculation unit 14 calculates the weight Λ(ind2) corresponding to Yᵢₙᵢ(ind2) having a value closest to Yₖ at the control time k out of Yᵢₙᵢ stored in the weight DB 22 as the weight R used to calculate the control value uₖ. Here, ind2 is an index of a time corresponding to Yᵢₙᵢ having a value closest to that of Yₖ. This corresponds to calculation of the weight R corresponding to the target data at the times k to k+h based on the past control results.

The weight calculation unit 14 transfers the calculated weight R to the control value calculation unit 15. Accordingly, as described above, the control value calculation unit 15 calculates the control value by using the weight R. The weight R is selected from the weights Λ stored in the weight DB 22, and the weights Λ are corrected by comparing the results of the target data with the target range. Thus, by calculating the control value with the weight R, control may be performed so as to confine the target data within the target range.

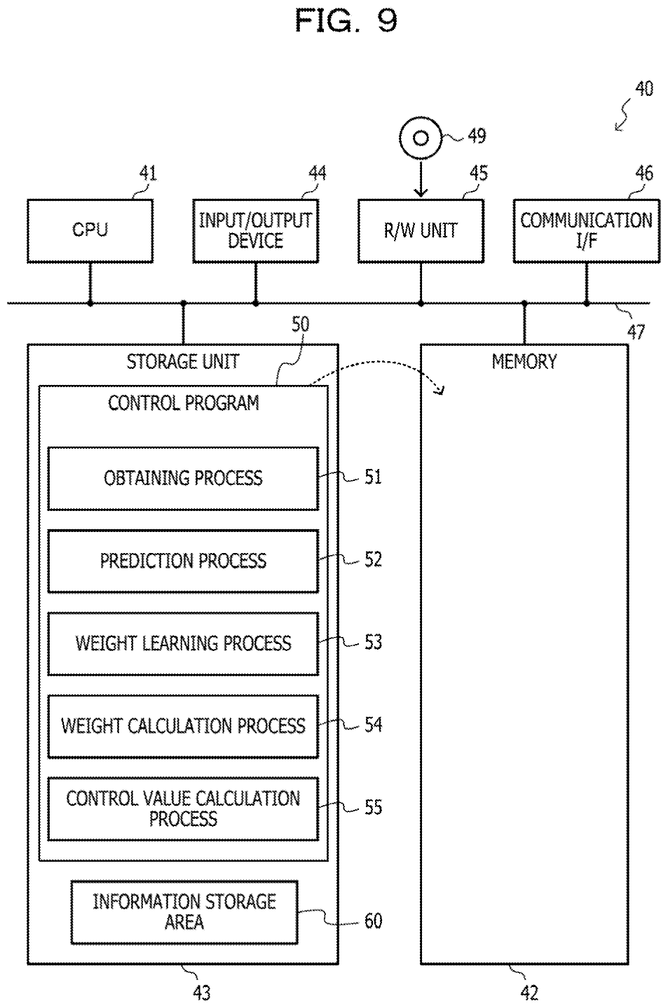

The control apparatus 10 is able to be realized by, for example, a computer 40 illustrated in FIG. 9. The computer 40 includes a central processing unit (CPU) 41, a memory 42 serving as a temporary storage area, and a nonvolatile storage unit 43. The computer 40 also includes an input/output device 44 such as an input unit, a display unit, and the like, and a read/write (R/W) unit 45 that controls reading and writing of data from and to a storage medium 49. The computer 40 also includes a communication interface (I/F) 46 that is coupled to a network such as the Internet. The CPU 41, the memory 42, the storage unit 43, the input/output device 44, the R/W unit 45, and the communication I/F 46 are coupled to each other via a bus 47.

The storage unit 43 may be realized by using a hard disk drive (HDD), a solid-state drive (SSD), a flash memory, or the like. The storage unit 43 serves as a storage medium that stores a control program 50 that causes the computer 40 to function as the control apparatus 10. The control program 50 includes an obtaining process 51, a prediction process 52, a weight learning process 53, a weight calculation process 54, and a control value calculation process 55. The storage unit 43 includes an information storage area 60 in which information configuring each of the prediction model 21 and the weight DB 22 is stored.

The CPU 41 reads the control program 50 from the storage unit 43, loads the control program 50 onto the memory 42, and sequentially executes the processes included in the control program 50. The CPU 41 executes the obtaining process 51 to operate as the obtaining unit 11 illustrated in FIG. 3. The CPU 41 executes the prediction process 52 to operate as the prediction unit 12 illustrated in FIG. 3. The CPU 41 executes the weight learning process 53 to operate as the weight learning unit 13 illustrated in FIG. 3. The CPU 41 executes the weight calculation process 54 to operate as the weight calculation unit 14 illustrated in FIG. 3. The CPU 41 executes the control value calculation process 55 to operate as the control value calculation unit 15 illustrated in FIG. 3. The CPU 41 reads information from the information storage area 60 and loads each of the prediction model 21 and the weight DB 22 onto the memory 42. In this way, the computer 40 executing the control program 50 functions as the control apparatus 10. The CPU 41 that executes the program is hardware.

The functions realized by the control program 50 may also be realized by, for example, a semiconductor integrated circuit, more in detail, an application-specific integrated circuit (ASIC) or the like.

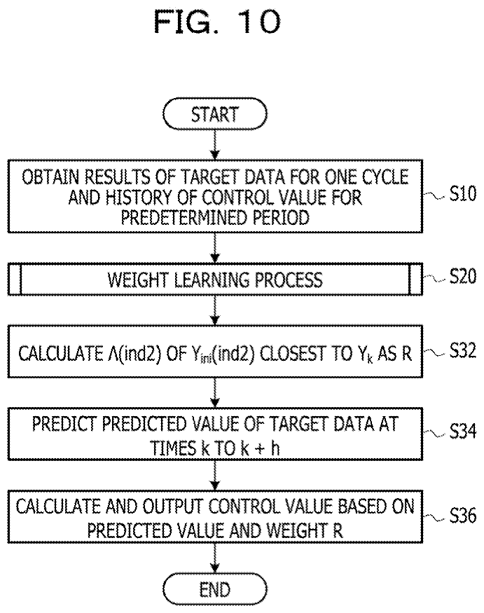

Next, operation of the control system 100 according to the present embodiment is described. When the measurement apparatus 30 starts the measurement and output of the target data, the control apparatus 10 executes a control process illustrated in FIG. 10 at each control time k. The control process is an example of a method of controlling of the disclosed technique. The control process is described below with the case, as an example, where control is performed such that the target data decreases as the control value increases as in the case of the blood glucose level control by insulin administration.

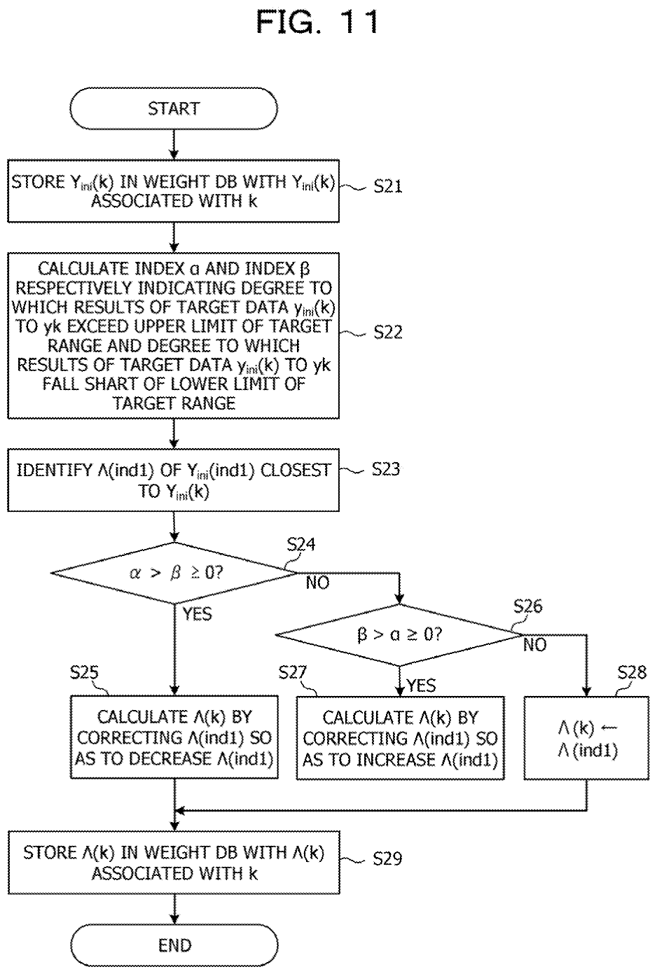

In step S10, the obtaining unit 11 obtains the results of the target data for a single cycle of a long-term prediction of the target data, for example, the results of the target data at each time from the time k−h to the time k and obtains the history of the control value for a predetermined period to the time k−h. Next, in step S20, the weight learning unit 13 executes the weight learning process. The weight learning process in step S20 is described with reference to FIG. 11.

In step S21, the weight learning unit 13 sets the result yᵢₙᵢ(k) of the target data at the time k−h out of the results of the target data obtained in step S10 above. The weight learning unit 13 stores in the weight DB 22 the vector value Yᵢₙᵢ(k) including values of yᵢₙᵢ(k) and the history uᵢₙᵢ(k) of the control value for a predetermined period to the time k−h in association with the index k of the time.

Next, in step S22, the weight learning unit 13 calculates, for a period from the time k−h to the time k, the index α indicating the degree to which the results (yᵢₙᵢ(k) to yₖ) of the target data exceed the upper limit of the target range and the index β indicating the degree to which the results (yᵢₙᵢ(k) to yₖ) of the target data fall short of the lower limit of the target range. Next, in step S23, the weight learning unit 13 identifies a weight Λ(ind1) corresponding to Yᵢₙᵢ(ind1) having a value closest to that of Yᵢₙᵢ(k) out of Yᵢₙᵢ stored in the weight DB 22.

Next, in step S24, the weight learning unit 13 determines whether β is greater than or equal to 0 and α is greater than β. For example, it is determined whether the degree of the results of the target data exceeding the upper limit of the target range is high. When α>β≥0, the process proceeds to step S25. When α≤β, the process proceeds to step S26. In step S25, the weight learning unit 13 calculates Λ(k) by correcting Λ(ind1) so as to decrease Λ(ind1) to increase the control value.

In step S26, the weight learning unit 13 determines whether α is greater than 0 and β is greater than α. For example, it is determined whether the degree of the results of the target data falling short of the lower limit of the target range is high. When β>α≥0, the process proceeds to step S27. When α=β=0, the process proceeds to step S28. In step S27, the weight learning unit 13 calculates Λ(k) by correcting Λ(ind1) so as to increase Λ(ind1) to decrease the control value.

In step S28, the weight learning unit 13 sets Λ(ind1) as it is as Λ(k). Here, in the case of α=β>0, in the case where there is a high risk when the target data exceeds the upper limit of the target range, it is sufficient that the weight learning unit 13 be predetermined to execute the process in step S25. In the case where there is a high risk when the target data falls short of the lower limit of the target range, the weight learning unit 13 may be predetermined to execute the process in step S27. When the risk in the case where the target data exceeds the upper limit and the risk in the case where the target data falls short of the lower limit are equal to each other, the weight learning unit 13 may execute the processing of step S28.

Next, in step S29, the weight learning unit 13 stores Λ(k) calculated in step S25, S27, or S28 described above in the weight DB 22 with Λ(k) associated with the index k of the time, ends the weight learning process, and returns to step S32 of the control process (FIG. 10).

Next, in step S32, the weight calculation unit 14 sets, as Yₖ, a vector value including values of the result yₖ of the target data at the control time k and the history u(k) of the control value for a predetermined period to the time k. The weight calculation unit 14 calculates the weight Λ(ind2) corresponding to Yᵢₙᵢ(ind2) having a value closest to Yₖ out of Yᵢₙᵢ stored in the weight DB 22 as the weight R used to calculate the control value uₖ. Next, in step S34, the prediction unit 12 predicts the predicted value xₖ₊ᵢ (i=0, 1, . . . , h) of the target data at each of the times k, k+1, . . . , k+h by using the results of the target data obtained in step S10 above and the control value calculated at the previous control time.

Next, in step S36, the control value calculation unit 15 calculates the control values uₖ₊ᵢ based on the predicted value xₖ₊ᵢ predicted in step S34 above and the weight R calculated in step S32 above. Out of the calculated control values uₖ₊ᵢ, the control value calculation unit 15 inputs the control value uₖ to the processing apparatus 32 to control the control target. The control value calculation unit 15 transfers the calculated control values uₖ₊ᵢ to the prediction unit 12 so that the calculated control values uₖ₊ᵢ are used for the long-term prediction of the target data at the next control time. The control value calculation unit 15 transfers the control value uₖ input to the processing apparatus 32 to the weight learning unit 13 as the history of the control value to be used in the weight learning process at the next and subsequent control times, and the control process ends.

As described above, with the control system according to the present embodiment, the control apparatus obtains the results of the target that fluctuates in accordance with the control by the system. The control apparatus calculates the weight for the control value in accordance with the history of the control value input to the system and the results and a comparison between the results and the specific range. Based on the results and the weight, the control apparatus calculates the control value, inputs the calculated control value to the system, and controls the target. Accordingly, even in the case where prediction of the target data using a prediction model is incorrect, control of confining the control target within the specific range may be adaptively performed without refining the prediction model.

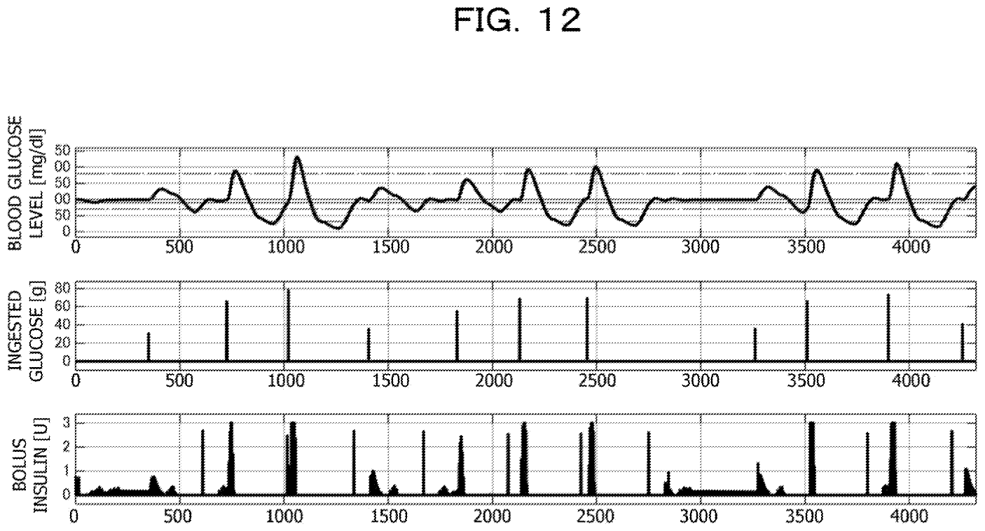

A control result when the disclosed technique is applied to the blood glucose level control by insulin administration is described. First, for comparison, FIG. 12 illustrates examples of the control results when learning or calculation of the weight for the control value is not performed. In FIG. 12, the blood glucose level is the example of the target data and the bolus insulin is the example of the control value. In FIG. 12, ingested glucose is the amount of glucose ingested by food and is a factor that affects fluctuation of the blood glucose level. The ingested carbohydrate amount in FIG. 2 described above affects similarly. When there is a factor that affects the fluctuation of the target data other than the control value as described above, it is sufficient that Expression (1) for the constraint of the prediction model described above be changed to an Expression to which a value zₖ of another factor is added as in Expression (10) below. D is a parameter matrix.

xₖ₊₁=Axₖ+Buₖ+Dzₖ (10)

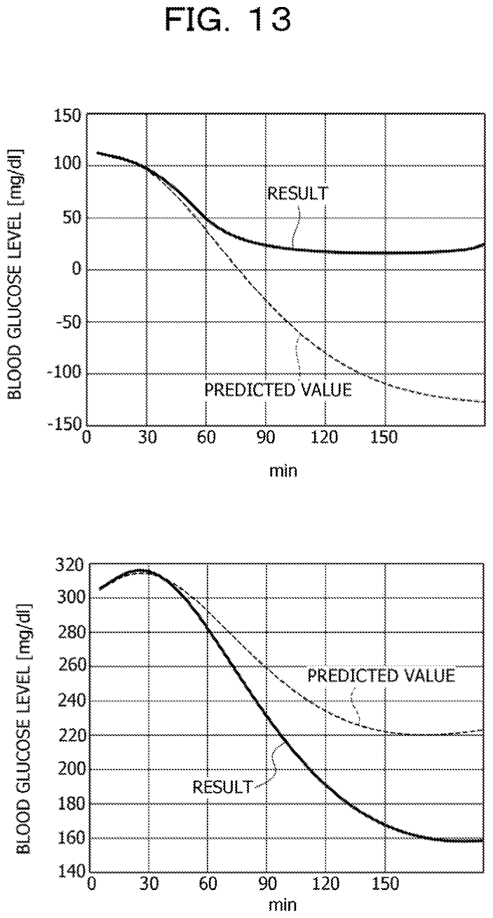

In FIG. 12, time periods in which the blood glucose level being the target data falls outside the target range (one dot chain lines in the upper diagram of FIG. 12), for example, falls short of the lower limit of the target range are frequently seen. FIG. 13 illustrates comparisons between the predicted value of the target data predicted at two different times and the result in the example illustrated in FIG. 12. At both of the times, there is a large difference between the predicted value and the result. It is thought that this difference causes the blood glucose level being the target data to fall outside the target range.

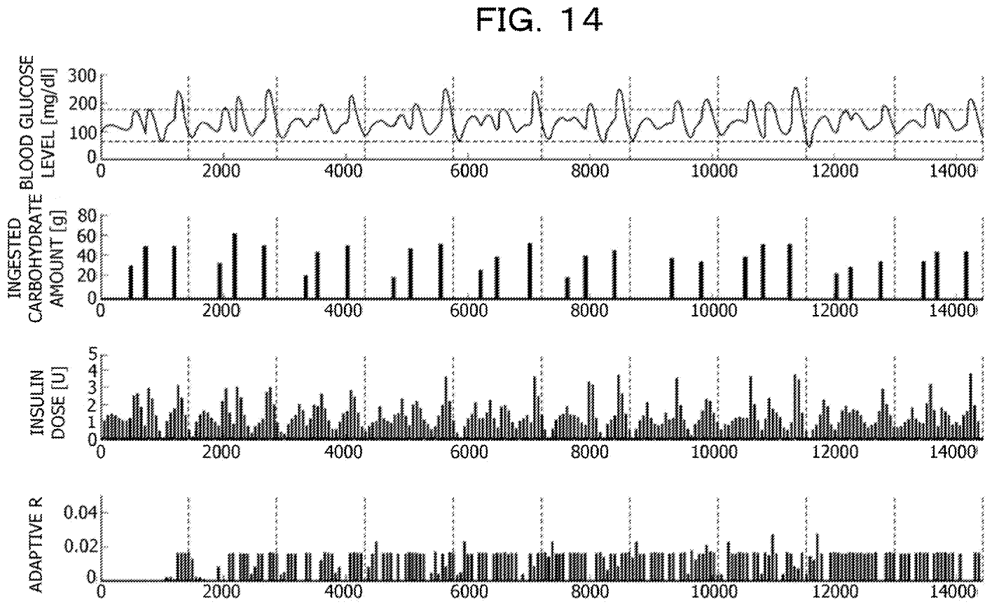

FIG. 14 illustrates examples of the control results when learning and calculation of the weight for the control value are performed as is the case with the present embodiment. Compared to the examples illustrated in FIG. 12, it may be understood that a time period in which the blood glucose level being the target data falls outside the target range (dashed lines in the upper diagram of FIG. 14) and the degree to which the blood glucose level falls outside the target range decrease.

Although the example of the blood glucose level control by insulin administration has been described for the case of the description of the application example according to the above-described embodiment, the disclosed technique is also applicable to another control system such as engine control.

Although a form has been described in which the control program is stored (installed) in a storage unit in advance according to the above-described embodiment, this is not limiting. The program according to the disclosed technique may be provided in a form in which the program is stored in a storage medium such as a compact disc read-only memory (CD-ROM), a Digital Versatile Disc (DVD)-ROM, or a Universal Serial Bus (USB) memory.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing a control program for causing a computer to execute a process comprising:
obtaining a result of a target that fluctuates in accordance with control performed by a system;
calculating a weight for a control value in accordance with the result, a history of the control value input to the system and a comparison between the result and a predetermined range;
calculating the control value based on the result and the weight; and
inputting the calculated control value to the system to control the target.

2. The non-transitory computer-readable recording medium according to claim 1, wherein
the calculating the weight includes calculating the weight that corresponds to the result obtained and a history of the control value inputted to the system based on a relationship between the history of the past result and the past control value and a weight that corresponds to the history of the past result and the past control value.

3. The non-transitory computer-readable recording medium according to claim 2, wherein
the relationship associates the history of the control value and the result, at a predetermined time, with the weight calculated based on results for a predetermined period from the predetermined time.

4. The non-transitory computer-readable recording medium according to claim 3, wherein,
The process further includes adding to the relationship, at each time at which the target is controlled, a correspondence of a result at a predetermined period preceding time that precedes a time at which the target is controlled and a history of the control value up to the predetermined period preceding time to a weight obtained by correcting, in accordance with a comparison between the predetermined range and the results for the predetermined period from the predetermined period preceding time, the weight associated with the result and the history of the control value values of which are closest to values of the result and the history of the control value at the predetermined period preceding time in the relationship.

5. The non-transitory computer-readable recording medium according to claim 4, wherein
the calculating the weight includes obtaining the weight associated with the result and the history of the control value in the relationship closest to the result and the history of the control value obtained this time.

6. The non-transitory computer-readable recording medium according to claim 1, wherein
the calculating the control value includes calculating the control value based on the weight and a predicted value of the target in accordance with the result.

7. The non-transitory computer-readable recording medium according to claim 6, wherein
the calculating the control value includes predicting the predicted value based on a model generated by machine learning, the result, and the control value.

8. The non-transitory computer-readable recording medium according to claim 6, wherein
the calculating the control value includes optimizing an objective function that includes a term for optimizing so as to confine the predicted value of the target within the predetermined range and a term for optimizing an input plan of the control value.

9. A control apparatus including:
a memory; and
a processor coupled to the memory and configured to execute a process comprising:
obtaining a result of a target that fluctuates in accordance with control performed by a system;
calculating a weight for a control value in accordance with the result, a history of the control value input to the system, and a comparison between the result and a predetermined range;
calculating the control value based on the result and the weight; and
inputting the calculated control value to the system to control the target.

10. The control apparatus according to claim 9, wherein
the calculating the weight includes calculating the weight that corresponds to the result obtained and a history of the control value inputted to the system based on a relationship between the history of the past result and the past control value and a weight that corresponds to the history of the past result and the past control value.

11. The control apparatus according to claim 10, wherein
the relationship associates the history of the control value and the result, at a predetermined time, with the weight calculated based on results for a predetermined period from the predetermined time.

12. The control apparatus according to claim 11, wherein,
the process further includes adding to the relationship, at each time at which the target is controlled, a correspondence of a result at a predetermined period preceding time that precedes a time at which the target is controlled and a history of the control value up to the predetermined period preceding time to a weight obtained by correcting, in accordance with a comparison between the predetermined range and the results for the predetermined period from the predetermined period preceding time, the weight associated with the result and the history of the control value values of which are closest to values of the result and the history of the control value at the predetermined period preceding time in the relationship.

13. The control apparatus according to claim 12, wherein
the calculating the weight includes obtaining the weight associated with the result and the history of the control value in the relationship closest to the result and the history of the control value obtained this time.

14. The control apparatus according to claim 9, wherein
the calculating the control value includes calculating the control value based on the weight and a predicted value of the target in accordance with the result.

15. The control apparatus according to claim 14, wherein
the calculating the control value includes predicting the predicted value based on a model generated by machine learning, the result, and the control value.

16. The control apparatus according to claim 14, wherein
the calculating the control value includes optimizing an objective function that includes a term for optimizing so as to confine the predicted value of the target within the predetermined range and a term for optimizing an input plan of the control value.

17. A method of controlling a system by a computer, the method comprising steps of:
obtaining a result of a target that fluctuates in accordance with control performed by a system;
calculating a weight for a control value in accordance with the result, a history of the control value input to the system and a comparison between the result and a predetermined range;
calculating the control value based on the result and the weight; and
inputting the calculated control value to the system to control the target.

18. The method of controlling according to claim 17, wherein
the calculating the weight includes calculating the weight that corresponds to the result obtained based on a relationship between a past result and a weight that corresponds to the past result.

19. The method of controlling according to claim 18, wherein
the relationship associates the result at a predetermined time, with the weight calculated based on results for a predetermined period from the predetermined time.
